(19) United States
(12) Patent Application Publication
(10) Pub. No.: US 2018/0177632 A1
(43) Pub. Date: Jun. 28, 2018

(54) SYSTEMS, METHODS, AND KITS TO REDUCE SURFACE HEATING DURING TISSUE TREATMENT

(71) Applicant: ALeyeGN Tehcnologies LLC, Saratoga, CA (US)

(72) Inventor: Satish Herekar, Palo Alto, CA (US)

(21) Appl. No.: 15/896,886

(22) Filed: Feb. 14, 2018

Related U.S. Application Data
(63) Continuation of application No. PCT/US2017/055408, filed on Oct. 5, 2017.
(60) Provisional application No. 62/538,549, filed on Jul. 28, 2017.
(60) Provisional application No. 62/404,422, filed on Oct. 5, 2016.

(51) Int. Cl. A61F 9/009; A61F 9/00

(52) U.S. Cl. CPC A61F 9/009; A61F 2009/00861; A61F 9/0017

(57) ABSTRACT

The disclosure generally relates to medical systems, devices and methods, and more particularly relates to dispersing heat during energy delivery to a tissue. The device may comprise two layers—a first layer which is optically transparent and a second later that may be both optically transparent and heat conductive. One or both of the layers may be configured to absorb energy (e.g., light energy), but together may transmit from about 50% to 99.9% of incident energy to the target tissue. One or both layers may comprise graphene or sapphire. One or both layers may comprise glass or plastic. The two layers may be any combination of glass, graphene, plastic, or sapphire. The two layers may be in physical contact with each other and either directly or indirectly bonded together.

### CROSS-REFERENCE

This application is a continuation of PCT application PCT/US2017/055408, entitled “SYSTEMS, METHODS, AND KITS TO REDUCE THERMAL INJURY DURING LASER EYE SURGERY” filed on Oct. 5, 2017, (attorney docket no. 48848-706.601), which claims the benefit of U.S. App. Ser. No. 62/404,422, entitled “SYSTEMS, METHODS, AND KITS TO REDUCE THERMAL INJURY DURING LASER EYE SURGERY” filed Oct. 5, 2016, (attorney docket no. 48848-706.101), and U.S. App. Ser. No. 62/538,549, entitled “SYSTEMS, METHODS, AND KITS TO REDUCE THERMAL INJURY DURING LASER EYE SURGERY” filed Jul. 28, 2017, (attorney docket no. 48848-706.102), which applications are entirely incorporated herein by reference. The subject matter of this patent application is also related to U.S. application Ser. No. 14/854,390, entitled “Scleral translocation elasto-modulation methods and apparatus” filed on Sep. 15, 2015, published as US20160113816A1, the entire disclosures of which are incorporated herein by reference.

### BACKGROUND OF THE INVENTION

Laser surgery of the eye can be used to treat the eye. However, heating of the eye can result in more heating of the eye than would be ideal. It would be beneficial to provide methods and apparatus of reducing heating of the eye during laser eye surgery. Although prior approaches have used materials to cool the eye, the prior materials can be bulky, more complicated or expensive to manufacture than would be ideal. Also, it would be helpful to have a single use consumable to facilitate a sterile environment. For example, contact with the eye has been used to reduce heating, the approaches can be more expensive to manufacture than would be ideal. Although sapphire has been previously proposed as a material to contact the eye, sapphire can have less than ideal mechanical and thermal conduction properties, and some of the prior sapphire eye contacting devices can be somewhat larger and more expensive to manufacture than would be ideal.

In light of the above, improved methods and apparatus for treated the eye are needed. Ideally, these would provide improved cooling of the eye, be easy to handle, compact, and be readily provided as single use consumable.

### SUMMARY

The present disclosure generally relates to dispersing heat during energy delivery to a tissue such as ocular tissues. Embodiments of the present disclosure will find application in many fields, where tissue is treated with energy. Although specific reference is made to a contact lens contacting an eye, the devices to conduct heat can be configured in many ways to treat non-ocular tissue with many types of energy such as ultrasound.

In many embodiments, a tissue coupling device, such as a lens, comprises an optically transmissive support material and a material comprising increased thermal conductivity such as a graphene. The tissue coupling device can be configured in many ways to conduct heat from the tissue with the optically transmissive support material and material comprising increased thermal conductivity. A layer comprising graphene can be placed proximity to the eye, in which the layer comprises sufficient amounts of graphene to conduct heat from the eye from focal regions of heating, such as a focused laser beam, in order to decrease surface heating of the tissue and increase heating of deeper regions of tissue. The layer comprising graphene may comprise a layer consisting of graphene, or a composite material comprising graphene another material. The composite material may comprise a mixture of graphene and plastic, such as a matrix comprising graphene and plastic. The tissue coupling device may comprise a portion with decreased amounts of graphene located away from a tissue contact surface and another portion with increased amounts of graphene near the tissue contact surface to increase thermal conductivity near the tissue contact surface. Alternatively or in combination, the tissue coupling device may comprise a thickness sufficient to provide strength while providing sufficient amounts of graphene near the tissue contact surface to readily conduct heat from the tissue surface. In some embodiments, the tissue coupling device may comprise a composite material having a thickness of no more than about 1 mm in order to provide appropriate amounts of heat conduction from the tissue. The tissue coupling device may comprise a single layer, such as a composite material comprising the optically transmissive support material and the thermally conductive material. The combination of the optically transmissive support material and thermally conductive material can allow the device to be manufactured easily and provided in single use sterile packaging.

The tissue coupling device can be configured in many ways, and may comprise a lens configured to contact an eye, such a meniscus lens, or a substantially flat plate configured to applanate the eye or contact other tissues and organs. The contact lens may comprise two layers—a first layer and a second layer which are optically transmissive and heat conductive, in which one of the layers has greater thermal conductivity than the other layer. One or both of the layers may be optically transmissive and configured to absorb some light energy, and together may transmit from about 50% to 99.9% of incident light energy to the target tissue. A first layer may comprise a support layer comprising a material such as plastic, for example poly carbonate or acrylate. A second layer may comprise a thin layer of highly conductive material to conduct heat away from the eye. The second layer can be very thin, such that the second layer may not be capable of freely standing without support of the first layer or another substrate. The two layers may comprise any combination of glass, graphene, plastic, sapphire or graphene like material. The two layers may be in physical contact with each other and either directly or indirectly bonded together.

The contact lens may operatively contact the human body when in use. For example, the contact lens may be in contact with the human eye in a manner similar to a contact lens. Energy (i.e. laser energy or high intensity focused ultrasound “HIFU” energy) may be applied to the eye through the contact lens. Heat generated by the energy may be dispersed by one or more of the layers of the contact lens to protect the surface of the eye from excessive heating. In many embodiments, the contact lens is configured to contact an epithelium of the eye and conduct absorbed by the eye away from the eye to decrease heating, such that the epithelium remains intact.

In one aspect, a lens for use in laser eye surgery is provided. In many embodiments, the lens comprises a first layer; and a second layer, the second layer supported with the first layer, the second layer thinner than the first layer, and the first layer comprises plastic and the second layer comprises graphene.

In another aspect, a lens for conducting heat away from an eye during laser eye surgery is provided. The lens comprises: a first surface for contacting the eye; a second surface for receiving light energy, wherein the lens comprises graphene between the first surface and the second surface and wherein the lens is configured to transmit light with a transmittance within a range from about 50% to 99%.

In some embodiments, the lens comprises a layer comprising graphene between the first surface and the second surface. In some cases, the layer comprising graphene comprises the first surface and the second surface and optionally wherein the first surface and the second surface comprise outer surfaces of the lens, the first surface comprising a posterior surface and the second surface comprising an anterior surface. In some cases, the layer comprises a cured mixture of graphene and an optically transmissive material located between the first surface and the second surface. In an example, second surface comprises a surface of the cured mixture. In another example, the cured mixture comprises a percentage of graphene within a range from about 1% to about 50% by weight and a percentage of optically transmissive material within a range from about 99% to about 50%. In another example, the cured mixture of graphene comprises a matrix comprising graphene particles dispersed in the optically transmissive material and optionally wherein the graphene particles are dispersed in the optically transmissive material with a uniformity of +/−5%. In a further example, the cured mixture of graphene comprises a matrix comprising graphene particles dispersed in the optically transmissive material and wherein an absorbance of the matrix varies by no more than +/−5% for a light beam transmitted through the matrix.

In another aspect, a lens for conducting heat away from an eye during laser eye surgery is provided. The lens comprises: a first layer comprising a first radius; and a second layer coupled to the first layer, and the second layer comprises a second radius within about 25% of the first radius, is thinner than the first layer, and comprises a thermally conductive material to conduct heat away from the eye in said laser eye surgery.

In some embodiments, the first layer comprises plastic and wherein the second layer comprises graphene. In some cases, the graphene comprises monolayer graphene sheets, bilayer graphene, graphene nanoribbons, graphene quantum dots, graphene oxide, chemically modified graphene, graphene ligand/complex, graphene fiber, 3D graphene pillared graphene, reinforced graphene, molded graphene, graphene aerogel, or graphene nanocoil.

In some embodiments, the lens further comprises a third layer configured to aid in preventing thermal damage to the eye. In some embodiments, the first layer comprises an optically transparent material. For example, the first layer comprises plastic and the plastic comprises one or more of acrylate, poly(methyl methacrylate) (PMMA), silicone, silicon elastomer, polycarbonate, polytetrafluoroethylene (PTFE), sapphire, or polyethylene terephthalate (PET).

In some embodiments, the lens comprises a thickness within a range of about 0.5 mm to 3.5 mm. In some cases, a portion of the lens comprising graphene comprises no more than 50% of the thickness of the lens or no more than 25% of the thickness of the lens. Alternatively, the lens comprises a thickness no more than about 0.5 mm or a thickness no more than about 3.5 mm. In some embodiments, the lens comprises a substantially uniform thickness across a distance of about 10 mm and optionally wherein the thickness varies by no more than 10% across the distance.

In some embodiments, the lens comprises an optical transmission within a range of about 50% to 99.9%. In some cases, the optical transmission is more than about 70% or is more than about 95%. In some embodiments, the second layer is configured to absorb about 0.1% to 25% of energy directed to the lens. In some embodiments, the lens is configured to receive energy with a wavelength from about 1.4 um to 1.6 um and/or with a wavelength from about 1.8 um to 2.2 um. In some embodiments, the second layer comprises a cured mixture comprising a percentage of graphene within a range from about 1% to about 50% by weight and a percentage of optically transmissive material within a range from about 99% to about 50%.

In some embodiments, the lens comprises a thermal conductivity greater than 40 W/mk. Alternatively, the lens comprises a thermal conductivity within a range from about 40 to 100 W/mK.

In some embodiments, the lens comprises no substantial optical power. In some embodiments, the lens comprises a convex anterior surface and a concave posterior surface when placed on the eye. Alternatively, the lens comprises a substantially flat anterior surface and a concave posterior surface when placed on the eye. In some embodiments, the lens comprises a biconvex lens, plano-convex lens, positive meniscus lens, negative meniscus lens, plano-concave lens, or biconcave lens. In some cases, the lens is flexible.

In some embodiments, the first layer comprises a thermal conductivity within a range of about 0 W/mK to about 30 W/mK. For instance, the first layer comprises a thermal conductivity no greater than about 0.5 W/mK. In some embodiments, the second layer comprises a thermal conductivity within a range of about 40 W/mK to 6000 W/mK. For instance, the second layer comprises a thermal conductivity within a range of about 1500 W/mK to 2500 W/mK or in a range no greater than about 600 W/mK.

In some embodiments, the lens is a single use lens. In some cases, the lens comprises a barcode configured to be read by a reader. For example, the lens is configured to be disabled from being used after being read a first time by the reader.

In another aspect, a method of conducting heat away from an eye in laser eye surgery is provided. The method comprises: coupling a lens to an eye, and the lens comprising: a first surface for contacting the eye; a second surface for receiving light energy, wherein the lens comprises graphene between the first surface and the second surface and wherein the lens is configured to transmit light with a transmittance within a range from about 50% to 99%; directing energy to the eye using a laser; and conducting heat away from the eye via the graphene between the first surface and the second surface.

In some embodiments, the lens comprises a layer comprising graphene between the first surface and the second surface. In some cases, the layer comprising graphene comprises the first surface and the second surface and optionally wherein the first surface and the second surface comprise outer surfaces of the lens, the first surface comprising a posterior surface and the second surface comprising an anterior surface. In some embodiments, the layer comprises a cured mixture of graphene and an optically transmissive material located between the first surface and the second surface. In some cases, second surface comprises a surface of the cured mixture. In some cases, the cured mixture comprises a percentage of graphene within a range from about 1% to about 50% by weight and a percentage of optically transmissive material within a range from about 50% to about 99%. In some cases, the cured mixture of graphene comprises a matrix comprising graphene particles dispersed in the optically transmissive material and optionally wherein the graphene particles are dispersed in the optically transmissive material with a uniformity of +/−5%. In some cases, the cured mixture of graphene comprises a matrix comprising graphene particles dispersed in the optically transmissive material and wherein an absorbance of the matrix varies by no more than +/−5% for a light beam transmitted through the matrix.

In another aspect, a system for conducting laser eye surgery is provided. The system comprises: an energy source; and a lens comprising graphene. In some embodiments, the lens is a single use lens utilized in a single treatment session. In some embodiments, the energy source comprises a laser. In some embodiments, the energy source comprises a high intensity focused ultrasound (HIFU).

In some embodiments, the graphene comprises monolayer graphene sheets, bilayer graphene, graphene nanoribbons, graphene quantum dots, graphene oxide, chemically modified graphene, graphene ligand/complex, graphene fiber, 3D graphene pillared graphene, reinforced graphene, molded graphene, graphene aerogel, or graphene nanocoil. In some embodiments, the lens comprises a first layer comprising plastic and a second layer comprising the graphene. In some cases, the lens further comprises a third layer configured to aid in preventing thermal damage to the eye. In some cases, the first layer comprises an optically transparent material. In some cases, the plastic comprises one or more of acrylate, poly(methyl methacrylate) (PMMA), silicone, polycarbonate, polytetrafluoroethylene (PTFE), sapphire, or polyethylene terephthalate (PET), or any of their variants thereof

In some embodiments, the lens comprises a thickness within a range of about 0.5 mm to 3.5 mm. In some cases, the lens comprises a thickness no more than about 0.5 mm. In some cases, the lens comprises a thickness no more than about 3.5 mm. In some embodiments, the lens comprises a substantially uniform thickness across a distance of about 10 mm and optionally wherein the thickness varies by no more than 10% across the distance. In some embodiments, the lens comprises an optical transmission within a range of about 50% to 99.9%. In some cases, the optical transmission is more than about 70% or is more than about 95%. In some cases, the second layer is configured to absorb about 0.1% to 25% of energy directed to the lens. In some embodiments, the lens is configured to receive energy with a wavelength from about 1.4 um to 1.6 um or with a wavelength from about 1.8 um to 2.2 um. In some cases, the second layer comprises a cured mixture comprising a percentage of graphene within a range from about 1% to about 50% by weight and a percentage of optically transmissive material within a range from about 99% to about 50%.

In some embodiments, the lens comprises a thermal conductivity greater than 40 W/mk. In some embodiments, the lens comprises a thermal conductivity within a range from about 40 to 100 W/mK. In some embodiments, the lens comprises no substantial optical power. In some embodiments, the lens comprises a convex anterior surface and a concave posterior surface when placed on the eye. Alternatively, the lens comprises substantially flat anterior and posterior surfaces when placed on the eye. In some embodiments, the lens comprises a biconvex lens, plano-convex lens, positive meniscus lens, negative meniscus lens, plano-concave lens, or biconcave lens. In some cases, the lens is flexible.

In some embodiments, the first layer comprises a thermal conductivity within a range of about 0 W/mK to about 30 W/mK. For instance, the first layer comprises a thermal conductivity no greater than about 0.5 W/mK. In some embodiments, the second layer comprises a thermal conductivity within a range of about 40 W/mK to 6000 W/mK. For instance, the second layer comprises a thermal conductivity within a range of about 1500 W/mK to 2500 W/mK or in a range no greater than about 600 W/mK.

In another aspect, a device for conducting heat from tissue comprises an optically transmissive support material and a thermally conductive material. The thermally conductive material comprises a thermal conductivity greater than the optically transmissive material. A first surface is located on a first side of the optically transmissive support material for contacting a surface of the tissue. A second surface is located on a second side of the optically transmissive support material for receiving light energy. The thermally conductive material is located between a first location of the first surface and a second location of the second surface, and transmittance from the second location through the support material and the thermally conductive material to the first location on the first surface is within a range from about 50% to 99%.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1G illustrate cross-sections of contact lens devices with heat dispersing properties, in accordance with some embodiments.

FIGS. 2A-2G illustrate exemplary systems for holding a contact lens device at a subject's eye, in accordance with some embodiments.

FIG. 3 illustrates a kit comprising a contact lens, in accordance with some embodiments.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

The methods, lenses, apparatus, devices and systems described herein may be well suited for combination with many types of treatments. The contact lenses disclosed herein may be well suited for combination with prior contact lenses in order to provide contact lenses with improved heat conduction and surface strength. For example, although reference is made to contact lenses, features of the presently disclosed lenses can be combined with spectacles to provide lenses having increased surface strength and scratch resistance. Also, features of the presently disclosed lenses are well suited for combination with many surgical devices, such as endoscopic surgery, and the thermally conductive layers as disclosed herein can be incorporated into endoscopic and other such as laser based endoscopic treatments.

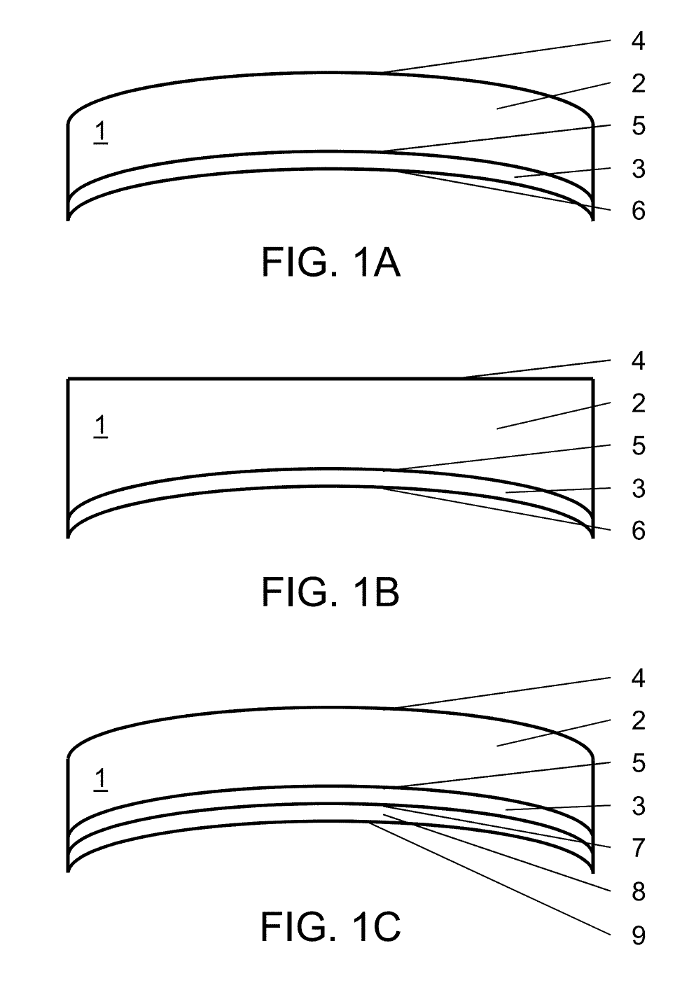

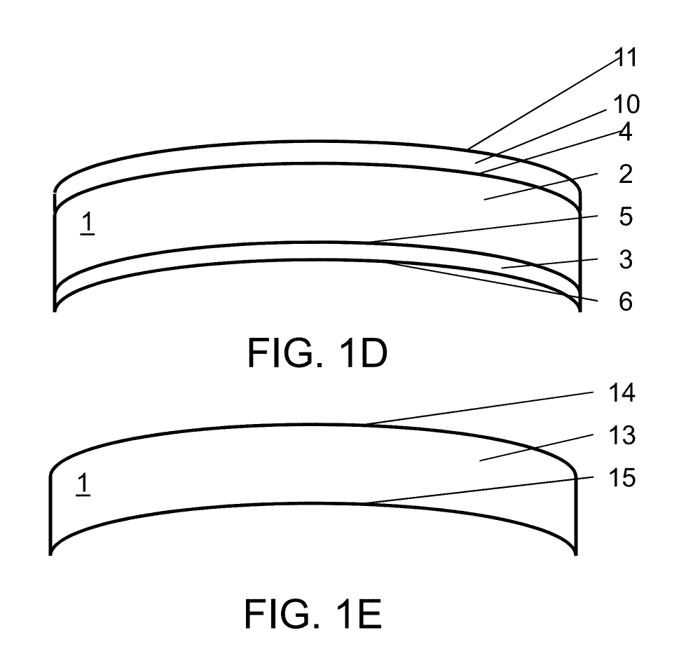

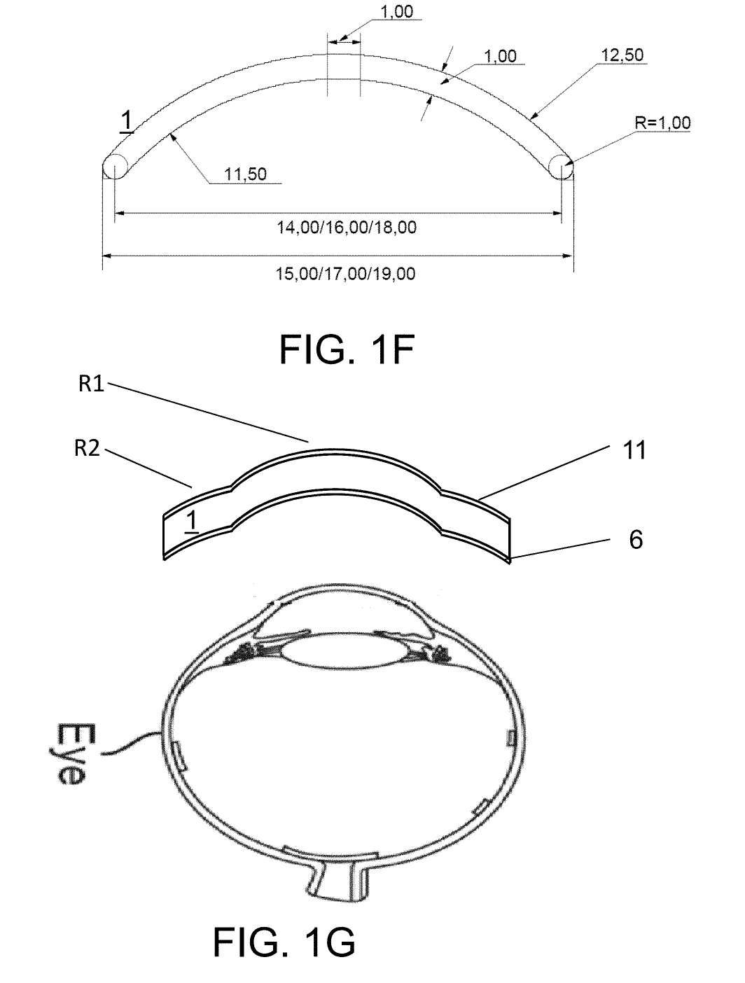

FIGS. 1A-1E show cross-sections of exemplary embodiments of contact lens devices 1 with heat dispersing properties.

FIG. 1A shows a contact lens 1 comprising a first layer 2 with anterior surface 4 and a posterior surface and a second layer 3 with an anterior surface and a posterior surface 6. The posterior surface of the first layer 2 and the anterior surface of the second layer 3 may be coupled at an interface 5. The posterior surface 6 of the second layer 3 may be shaped to substantially conform to the profile of the human eye. The posterior surface of the second layer 3 may comprise a radius of curvature from about 7 millimeters to about 13 millimeters (mm). Optionally, the posterior surface of the second layer 3 may comprise a radius of curvature equal to about 3 mm, 5 mm, 7 mm, 9 mm, 11 mm, 13 mm, 15 mm, 17 mm, 19 mm, 21 mm, or any value therebetween. The posterior surface of the second layer 3 may comprise two or more radii of curvature each with values from about 7 millimeters to about 13 millimeters. Optionally, the posterior surface of the second layer 3 may comprise two or more radii of curvature each equal to about 3 mm, 5 mm, 7 mm, 9 mm, 11 mm, 13 mm, 15 mm, 17 mm, 19 mm, 21 mm, or any value therebetween. An exemplary embodiment comprising two radii of curvature is shown in FIG. 1G. The contact lens may comprise a biconvex lens, plano-convex lens, positive meniscus lens, negative meniscus lens, plano-concave lens, or biconcave lens. In some embodiments, the thickness of the first layer 2 may be greater than the second layer 3. The first layer 2 and the second layer 3 may be constructed of different materials or different combination of materials.

The contact lens 1 may have any shape including but not limited to a circle, an oval, or an ellipse. The contact lens 1 may have a maximum thickness no greater than about 5 mm, 4.5 mm, 4 mm, 3.5 mm, 3 mm, 2.5 mm, 2 mm, 1.5 mm, 1 mm, 0.5 mm, or 0.25 mm. Optionally, the thickness can be within a range from about 0.5 mm to about 3.5 mm. The thickness of the contact lens 1 may vary across the contact lens 1 such that a first portion of the contact device may be up to about 10, 8, 6, 4, or 2 times thicker than a second portion of the device. Alternatively, the thickness of the contact lens 1 may be uniform across the device.

The contact lens 1 of this or any other embodiments described herein may have an energy transmittance from about 50% to about 99.9%. Optionally, the energy transmittance of the contact lens may be about 50%, 55%, 60%, 65%, 70%, 75%, 80%, 85%, 90%, 95%, 96%, 97%, 98%, 99%, or 99.9%, and within a range defined by any two of the aforementioned values. For example, the energy transmittance of the contact lens may be from about 50% to about 90%, or from about 50% to about 99%. In some instances, the contact lens described herein may have transmissive losses between 1-10%. Optionally, the contact lens may have transmissive losses equal to or less than about 1%, 2%, 3%, 4%, 5%, 6%, 7%, 8%, 9%, 10%, 11%, 12%, 13%, 14%, or 15%. In some instances, a thickness of the contact lens may have an effect on the transmissive losses. For example, the contact lens may have transmissive losses between 1-10% wherein the second layer 4 may be about 2 micrometers thick. The enhanced energy transmittance may be beneficial for the contact lens 1 to withstand energy. In some instances, the enhanced energy transmittance may be beneficial for the contract lens to withstand energy having a wavelength equal to about 0.4 um, 0.6 um, 0.8 um, 1 um, 1.2 um, 1.4 um, 1.6 um, 1.8 um, 2.0 um, 2.2 um, 2.4 um, 2.6 um, or any value therebetween. In some instances, the enhanced energy transmittance may be beneficial for the contract lens to withstand energy having a wavelength from about 1.4 um to 1.6 um or from about 1.8 um to 2.2 um during laser surgery. For example, the energy transmittance of the contact lens may be about at least 90%, 80%, 70%, 60% or 50% for wavelength around 2 um, at least 90%, 80%, 70%, 60% or 50% for wavelength around 1.8 um, at least 90%, 80%, 70%, 60% or 50% for wavelength around 2.2 um; the energy transmittance of the contact lens may be about at least 90%, 80%, 70%, 60% or 50% for wavelength around 1.4 um, at least 90%, 80%, 70%, 60% or 50% for wavelength around 1.5 um, and at least 90%, 80%, 70%, 60% or 50% for wavelength around 1.6 um.

The contact lens 1 may have thermal conductivity of greater than about 3000 W·m⁻¹·K⁻¹. The thermal conductivity of the contact lens 1 may be from about 1,500 W·m⁻¹·K⁻¹ to about 3,500 W·m⁻¹·K⁻¹. In some embodiments, the thermal conductivity of the contact lens 1 may be within a range from about 40 W·m⁻¹·K⁻¹ to about 6000 W·m⁻¹·K⁻¹.

The contact lens 1 may have an energy transmittance within a range from about 50% to about 99.9%. The light energy transmittance of the contact lens of any embodiments may be about 50%, 55%, 60%, 65%, 70%, 75%, 80%, 85%, 90%, 95%, 96%, 97%, 98%, 99%, or 99.9% and may be within a range defined by any two aforementioned values. In some embodiments, the contact lens 1 may have an optical transmittance of at least 80% at wavelength in a range of about 1 micrometer to 2.3 micrometers. The contact lens may absorb about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or about 50%, and may be within a range defined by any two of the aforementioned values. In some instances, the enhanced energy transmittance may be beneficial for the contract lens to withstand energy having a wavelength equal to about 0.4 um, 0.6 um, 0.8 um, 1 um, 1.2 um, 1.4 um, 1.6 um, 1.8 um, 2.0 um, 2.2 um, 2.4 um, 2.6 um, or any value therebetween. In some instances, the enhanced energy transmittance may be beneficial for the contract lens to withstand energy having a wavelength from about 1.4 um to 1.6 um or from about 1.8 um to 2.2 um during laser surgery.

In some embodiments, the contact lens 1 may not comprise substantial optical power. Optical power may refer to the degree to which a lens converges or diverges light. The contact lens 1 may not substantially diverge or converge light such as lasers passing through the contact lens. This is beneficial for laser surgeries where an accurate treatment location in the eye is identified from an image of the eye without substantial optical distortion caused by the contact lens. In alternative cases, the contact lens 1 may comprise optical power such that the treatment laser maybe directed to or concentrated in a desired location when passing through the contact lens.

In some embodiments, the contact lens 1 may be flexible. Alternatively, the contact lens 1 may be rigid.

In some embodiments, at least one layer of the contact lens 1 comprising graphene may be in contact with an eye. In some cases, use of graphene may assist in generating H₂O₂ which is a collagen crosslinker. Due to high electrical conductivity and high hydrophobicity, H₂O₂ may be generated and providing a continuous source of free radicals at the contact surface between the contact lens and an eye which can be titrated to the desired degree of corneal collagen crosslinking.

The first layer 2 of this or any embodiment may be a transmissive layer such that a substantial amount of energy (e.g., laser energy) delivered to its anterior surface 4 may be delivered through its posterior surface. The first layer 2 of this or any embodiment may comprise plastic. The first layer 2 of this or any embodiment may comprise a material such as acrylate, poly(methyl methacrylate) (PMMA), silicone, polycarbonate, polytetrafluoroethylene (PTFE), sapphire, or polyethylene terephthalate (PET), or any of their variants (e.g., PET-G). The first layer 2 of this or any embodiment may comprise a composite material of any combination of any of the aforementioned materials. In some cases, the first layer 2 may have a thickness of no more than 3 millimeters. In some cases, the first layer 2 may have a thickness of no greater than about 1 millimeter. In some cases, the first layer 2 may have a thickness of no greater than about 0.5 millimeters.

The first layer 2 of this or any embodiments may have an energy transmittance from about 50% to about 99.9%. The energy transmittance of the first layer 2 of any embodiments may be about 50%, 55%, 60%, 65%, 70%, 75%, 80%, 85%, 90%, 95%, 96%, 97%, 98%, 99%, or 99.9% or be within a range defined by any two of the aforementioned values, e.g. it may take any value between any two aforementioned values. In some cases, the first layer may comprise an optical transmission within a range of about 50% to 99.9%. The first layer may have an optical transmission of about 80%-99% in the mid IR (infrared) spectrum. The optical transmission of the first layer may be about 75%, 80%, 85%, 90%, 95%, 99%, and within a range defined by any two of the aforementioned values. For instance, the first layer may have an optical transmission of about 85% at wavelength of about 1.5 um.

The first layer 2 of this or any embodiment may have thermal conductivity of greater than about 30 W·m⁻¹·K⁻¹. The thermal conductivity of the first layer 2 may be no greater than about 0.5 W·m⁻¹·K⁻¹. In some embodiments, the thermal conductivity of the contact lens 1 may be within a range from about 0 to 30 W·m⁻¹·K⁻¹. In some embodiments, the thermal conductivity of the first layer 2 may be lower than the thermal conductivity of the second layer 3.

The second layer 3 may comprise graphene, although other materials with heat conduction and optical transmission properties similar to graphene can be used. The graphene may be of any form including but not limited to monolayer sheets, bilayer graphene, graphene nanoribbons, graphene quantum dots, graphene oxide, chemically modified graphene, graphene ligand/complex, graphene fiber, 3D graphene pillared graphene, reinforced graphene, molded graphene, graphene aerogel, or graphene nanocoil. The second layer 3 comprised of graphene may be produced in any manner known in the art including but not limited to exfoliation, hydrothermal self-assembly, chemical vapor deposition (CVD), nanotude slicing, carbon dioxide reduction, spin coating, sonic sprays, lasers, microwave-assisted oxidation, or ion implantation. In some embodiments, the second layer 3 may be transferred to the first layer 2 with a known transfer process, for example as described by ACS Material (on the Internet at acsmaterial.com). In some cases, the second layer may comprise a cured mixture comprising a percentage of graphene within a range from about 1% to about 50% by weight and a percentage of optically transmissive material within a range from about 99% to about 50%. In some cases, the second layer may absorb about 0.4% mid-IR range spectrum due to Pauli blocking. In the case when the second layer comprises a single layer graphene, the optical transmission may be about 97% at wavelength of about 2 um.

In the case when the second layer is a single layer graphene, bilayer graphene, or multiple layers, thermal conductivity and/or optical transmission of the second layer may be slightly different depending on the specific fabrication techniques (e.g., exfoliated or chemical vapor deposition (CVD) grown) used to prepare the graphene layer. The method and process for preparing a single layer CVD graphene is known to in the art. For instance, the process may comprise the precursor pyrolysis of a material to form carbon, and the formation of the carbon structure of graphene using the disassociated carbon atoms on a metal surface. In some cases, the process may also comprise a step to transfer from the metal surface to the target substrates (i.e. first layer). Various approaches may be used to transfer the formed CVD graphene to the target surface, such as using a carrier film, stamp method or self-release transfer method.

The first layer 2 and the second layer 3 may be in physical contact with each other and either directly or indirectly bonded together. In some embodiments, the two layers may be directly bonded together. For instance, the second layer 3 may be coated onto the first layer 2. In some embodiments, the two layers may be indirectly bonded together. For instance, materials or fillers such as pY-PGMA graphene epoxy or the like may be used at the interface of the two layers for efficient bonding.

In some cases, the second layer 3 may have a thickness of no more than about 0.5 millimeters. In some cases, the second layer 3 may have a thickness of no greater than about 0.05 millimeters. In some cases, the second layer 3 may have a thickness of no greater than about 0.005 millimeters (5 micrometers). In some embodiments, the second layer 3 may comprise a single layer of graphene such that the thickness may be no more than about 0.5 angstrom. The second layer 3 may have a dimeter or radius substantially equal to the first layer 2. Alternatively, the radius of the second layer 3 may be equal to or less than the radius of the first layer 2. The second radius can be within any percentage of the first radius. The second radius may be within about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 99.9%, and within a range defined by any two of the aforementioned values of the first radius. For example, the second layer may comprise a radius within about 25% of the radius of the first layer.

The second layer 3 may comprise any transparent conducting film now known in the art including but not limited to indium tin oxide, carbon nanotubes, transparent conductive oxides, ultrathin metal films. Alternatively or in combination, the second layer 3 may comprise acrylate, poly(methyl methacrylate) (PMMA), silicone, polycarbonate, polytetrafluoroethylene (PTFE), sapphire, or polyethylene terephthalate (PET), or any of their variants (e.g., PET-G). The second layer 3 may comprise plastic.

The percentage of graphene comprised by the second layer 3 may be around 70%. The percentage of graphene comprised by the second layer 3 may be in a range of 10%-90%. Material properties such as thermal and optical properties of the second layer 3 may be affected by the percentage of the graphene. In some cases, the second layer may comprise a cured mixture comprising a percentage of graphene within a range from about 1% to about 70% by weight.

The thermal conductivity of the second layer 3 may be no greater than about 5,300 W·m⁻¹·K⁻¹. The thermal conductivity of the second layer 3 may be from about 1,500 W·m⁻¹·K⁻¹ to about 3,500 W·m⁻¹K⁻¹. In some embodiments, the thermal conductivity of the second layer 3 may be at least about 3000 W·m⁻¹·K⁻¹. The second layer may comprise a thermal conductivity within a range of about 40 W·m⁻¹·K⁻¹ to 6000 W·m⁻¹·K⁻¹. The second layer may comprise a thermal conductivity within a range of about 1500 W·m¹·K¹ to 2500 W·m¹·K¹. The thermal conductivity of the second layer may be affected by the percentage of graphene.

The second layer 3 of may have an energy transmittance within a range from about 50% to about 99.9%. The light energy transmittance of the second layer 3 of any embodiments may be about 50%, 55%, 60%, 65%, 70%, 75%, 80%, 85%, 90%, 95%, 96%, 97%, 98%, 99%, or 99.9% and may be within a range defined by any two aforementioned values. The second layer3 may absorb about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, or about 50%, and may be within a range defined by any two of the aforementioned values. The optical transmittance of the second layer 3 may be affected by the percentage of graphene in the second layer. For example, the second layer with greater graphene percentage may have a lower optical transmittance.

The second layer 3 may absorb about 0.1% to 25% of energy directed to the lens. The purpose of the second layer is to transfer energy and heat away from the region to prevent undesirable amounts of heating to the eye.

The first layer 2 and the second layer 3 may be created simultaneously or sequentially. The second layer 3 may be deposited as a coating on the first layer 2.

FIG. 1B shows a cross-section of an exemplary embodiment of a contact lens 1 comprising a first layer 2 and a second layer 3, the first layer having an anterior surface 4 that is flat and a posterior surface with a shared interface 5 with the anterior surface of the second layer 3. The second layer's 3 posterior surface 6 may be of shape described herein. The flat anterior surface 4 may aid in transmitting, absorbing, or reflecting energy (e.g., laser energy) input to the contact lens 1. The anterior surface 4 may have microsurface features to aid in the transmission, absorption or reflection of energy. Though the embodiment illustrated in FIG. 1B may comprise a flat anterior surface 4 and a curved posterior surface 6, other embodiments may also be possible including but not limited to: a curved anterior surface 4 and a curved posterior surface 6; a curved anterior surface 4 and a flat posterior surface 6; a flat anterior surface 4 and a curved posterior surface 6 (as illustrated); a flat anterior surface 4 and a flat anterior surface 6; and a convex anterior surface and a concave posterior surface when placed on the eye.

FIG. 1C shows a cross-section of an exemplary embodiment of a contact lens 1 with heat dispersing properties comprising a first layer 2 with an anterior surface 4 and a posterior surface, a second layer 3 with an anterior surface and a posterior surface, and a third layer 8 with an anterior surface and a posterior surface 9, wherein a third layer 8 is disposed between the second layer 3 and a patient's eye (not illustrated). The third layer 8 may have a posterior surface 9 and an anterior surface with a shared interface 7 with the posterior surface of the second layer 3. The third layer 8 may comprise acrylate, poly(methyl methacrylate) (PMMA), silicone, polycarbonate, polytetrafluoroethylene (PTFE), sapphire, or polyethylene terephthalate (PET), or any of their variants (e.g., PET-G). The third layer 8 may aid in preventing thermal damage to the eye and/or may prevent irritation to the eye. In some cases, the third layer 8 may have a thickness of no more than about 1 millimeters. In some cases, the third layer 8 may have a thickness of no greater than about 0.5 millimeters. In some cases, the third layer 8 may have a thickness of no greater than about 0.1 millimeters.

FIG. 1D shows a cross-section of an exemplary embodiment of a contact lens 1 with heat dispersing properties comprising a first layer 2 with an anterior surface 4 and a posterior surface, a second layer 3 with an anterior surface and a posterior surface, and a third layer 10 with an anterior surface 11 and a posterior surface. The first layer 2 is disposed between the second layer 3 and the third layer 10. The third layer 10 may have an anterior surface 11 and a posterior surface with a shared interface 4 with the anterior surface of the first layer 2.

The third layer 10 may be a transmissive layer such that a substantial amount of energy (e.g., laser energy) delivered to its anterior surface 11 may be delivered through its posterior surface. The third layer 10 may comprise graphene. The third layer 10 may comprise acrylate, poly(methyl methacrylate) (PMMA), silicone, polycarbonate, polytetrafluoroethylene (PTFE), sapphire, or polyethylene terephthalate (PET), or any of their variants (e.g., PET-G). The third layer 10 may aid in preventing thermal damage to the eye. In some cases, the third layer 10 may have a thickness of no more than about 1 millimeter. In some cases, the third layer 10 may have a thickness of no greater than about 0.5 millimeters. In some cases, the third layer 10 may have a thickness of no greater than about 0.1 millimeters. The percentage of graphene in the second layer 3 and the third layer 10 may or may not be identical. In some embodiments, the percentage of graphene in the third layer 10 may be lower than the second layer 3. The percentage of graphene comprised by the third layer 10 may be in a range of 5%-50%.

In some embodiments, the first layer 2 may comprise a polymer material having sufficient stiffness to support the lens 1, the second layer 3 may comprise a thermally conductive layer, and the third layer 10 may comprise a thermally conductive layer. The first layer 2 may comprise a plastic material, such as PMMA, having sufficient stiffness to at least partially reshape the tissue of the eye when placed against the eye. The second layer 3 may comprise a thermally conductive material as described herein, such as a graphene monolayer. The first layer can be adhered to the third layer, and the first layer can support the third.

The lenses as described herein may comprise suitable optical properties to allow a surgeon to visualize the eye or other tissue through the lens during surgery. In general, the lenses comprise optical smoothness with low amounts of light scatter such that a surgeon can readily visualize tissues of the eye through an operating microscope, for example. The monolayers of graphene as described herein can provide optical uniformity and heat conduction and can be adhered to polymer surfaces such as plastic polymer surfaces. The graphene monolayers as described herein can transmit visible light uniformly to within about 10% over a range of visible wavelengths from about 400 to 800 nm, and with a transmittance of at least above 90%, for example. The inner layer of the lens may comprise optically smooth surfaces, and the layer of heat transmissive material on the second surface or the third surface may comprise a thickness less than visible light at 400 nm, for example a thickness within a range from about 3.5 Angstroms to about 400 nm, for example within a range from about 3.5 Angstroms to about 50 Angstroms. The outer layers of the lens (e.g. second and third layers) can be deposited in many ways as described herein.

The second layer 3 and optional third layer 10 may comprise sufficient surface area so as to conduct heat from tissue such as ocular tissue. Each of these layers may comprise a surface area within a range from about 10 mm² to about 300 mm². In some embodiments, the surface area of the highly conductive layer may be within a range from about 25 mm² to about 250 mm². The second layer 3 may comprise a concave external surface and a convex internal surface coupled to the first layer 2 and the third layer 10 may comprise a convex external surface and a concave internal surface coupled to the first layer 2. In some embodiments, the surface area of each layer corresponds to a sag height distance within a range from about 0.5 to 5 mm, for example within a range from about 1 to 4 mm. Each of the first layer, the second layer and the third layer may comprise optically transmissive materials as described herein, and each layer may comprise sufficient smoothness to form images through the lens corresponding to 20/20 vision (6/6 metric), for example corresponding to resolution to about 1 minute of arc. While the surfaces may be rougher, in many embodiments the root means square (RMS) of localized roughness (e.g. over about a 1 mm² area may be within a range from about 0.1 nm to 100 nm, and each of the layers may comprise an optically transmissive material as described herein.

FIG. 1E shows a contact lens 1 comprising a single layer 13 with anterior surface 14 and a posterior surface 15. The posterior surface 15 may be shaped to substantially conform to the profile of the human eye. The posterior surface may comprise a radius of curvature from about 7 millimeters to about 13 millimeters. The posterior surface may comprise two or more radii of curvature each with values from about 7 millimeters to about 13 millimeters.

The single layer contact lens may comprise graphene. The single layer 13 may be formed of a mixture of graphene and other materials such as acrylate, poly(methyl methacrylate) (PMMA), silicone, polycarbonate, polytetrafluoroethylene (PTFE), sapphire, or polyethylene terephthalate (PET), or any of their variants (e.g., PET-G). The single layer 13 may comprise materials substantially similar to the second layer 3 as described in FIG. 1A. The single layer 13 may have homogeneous clarity. The single layer 13 contact lens may comprise a composite of materials having a uniformity of less than 5% variation across the entire layer.

The contact lens may have varied overall dimensions or sizes to fit with different applications or patients. The contact lens may have an outer diameter of about 15 mm, 16 mm, 17 mm, 18 mm, 19 mm, or 20 mm. The heat sink contact lens may have an outer diameter of less than 15 mm. The heat sink contact lens may have an outer diameter of greater than 20 mm. The heat sink contact lens may have a thickness of about 0.5 mm, 1 mm, or 1.5 mm. The heat sink contact lens may have a thickness of less than 0.5 mm. The heat sink contact lens may have a thickness of greater than 1.5 mm. FIG. 1F schematically shows a contact lens having outer diameter of 15 mm, 17 mm, and 19 mm. In some embodiments, a radius of curvature may be varied in association with the outer diameter. For example, a radius of curvature may be increased with a greater outer diameter or size. Alternatively, the contact lens may have a constant radius of curvature across the different sizes or dimensions.

In some embodiments, the contact lens may comprise a posterior surface having two or more radii of curvature. This provides a posterior surface that conforms to the profile of human eye. FIG. 1G shows a contact lens 1 comprising an anterior surface 11 and a posterior surface 6. The posterior surface 6 may be shaped to substantially conform to the profile of the human eye. As shown in the drawings, the posterior surface may comprise two radii of curvature R1, R2 with values of about 7-8 millimeters and values of about 12-13 millimeters respectively. The values of the two radii of curvatures R1, R2 may or may not vary across different sizes or dimensions of the lens. The first radius of curvature R1 may meet the second radius of curvature at, for example, the location corresponding to limbus of an eye. As described elsewhere herein, the profile of the anterior surface 11 may or may not be the same as the posterior surface 6. Thus the thickness of the contact lens may or may not be uniform across the entire lens.

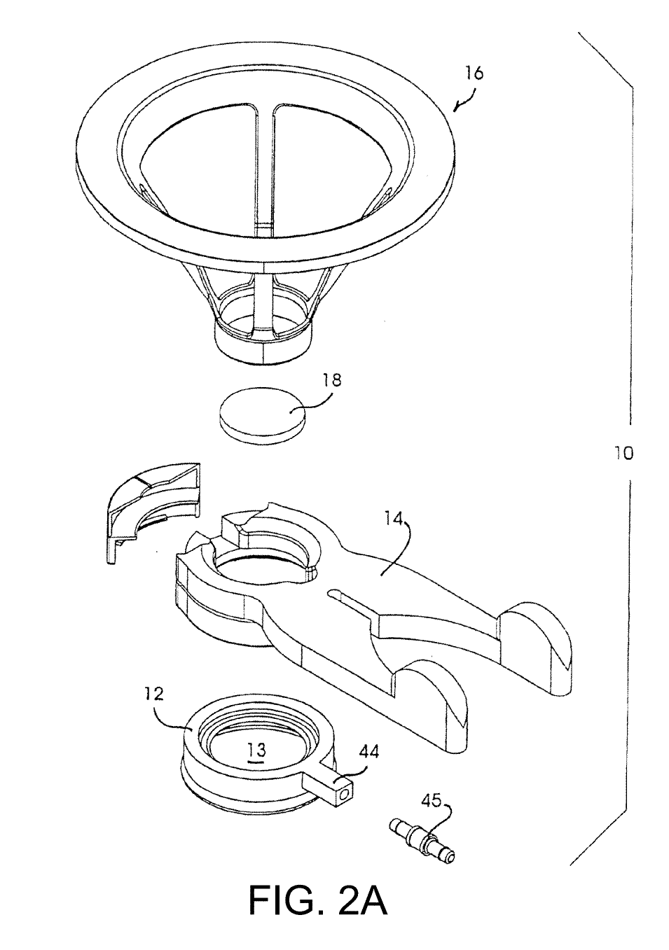

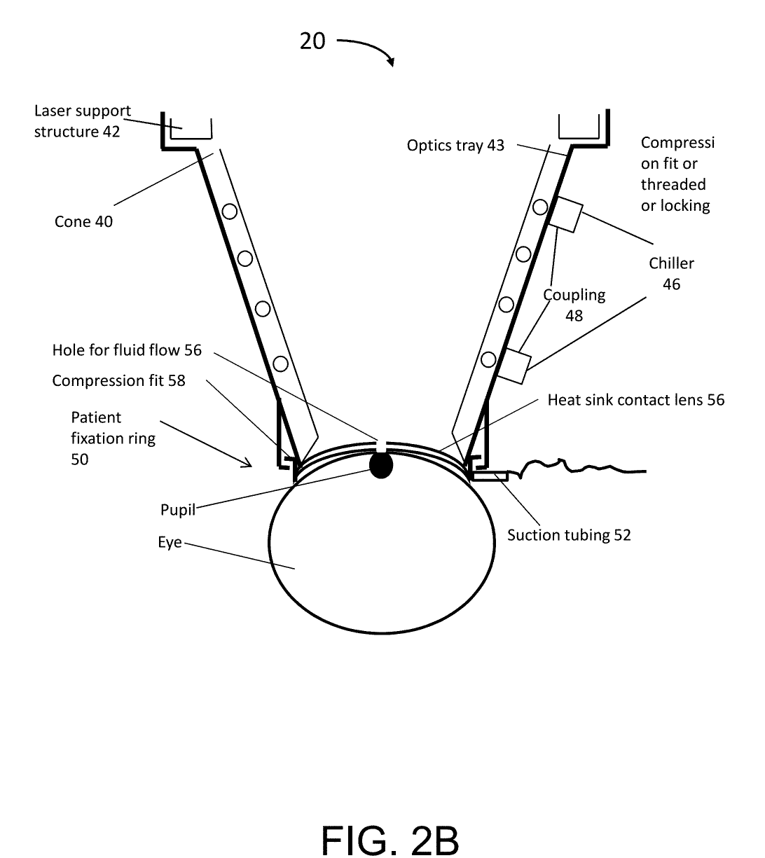

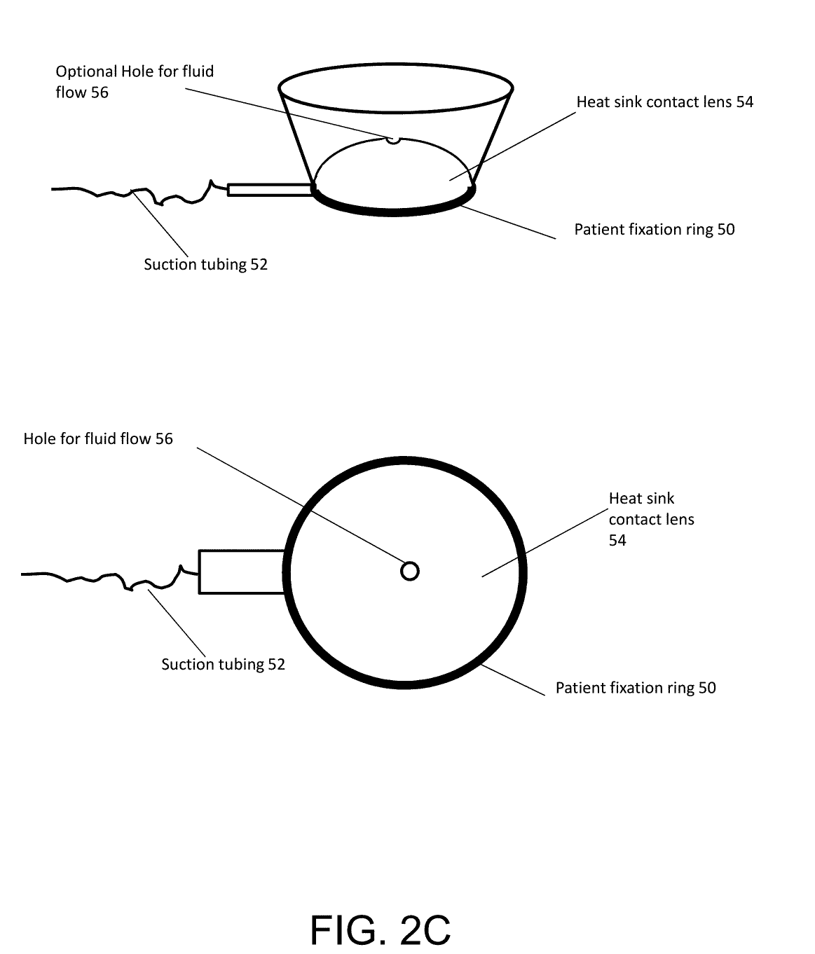

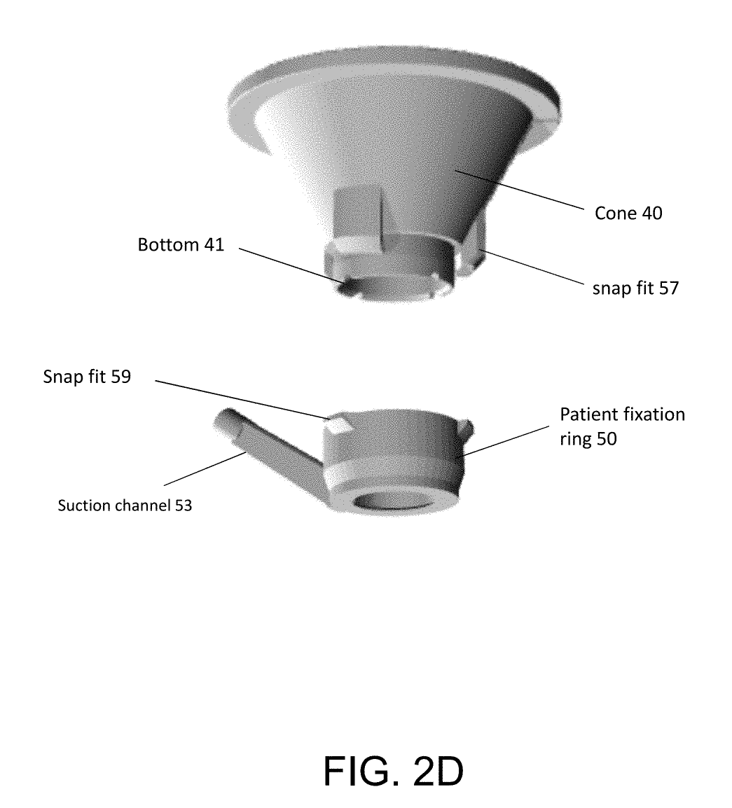

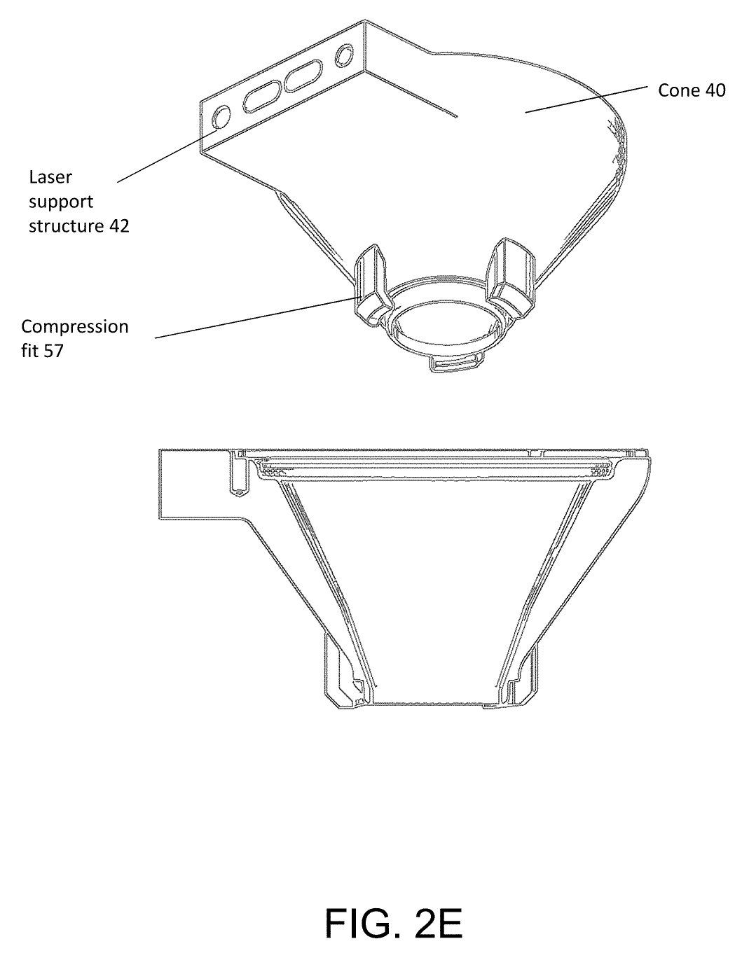

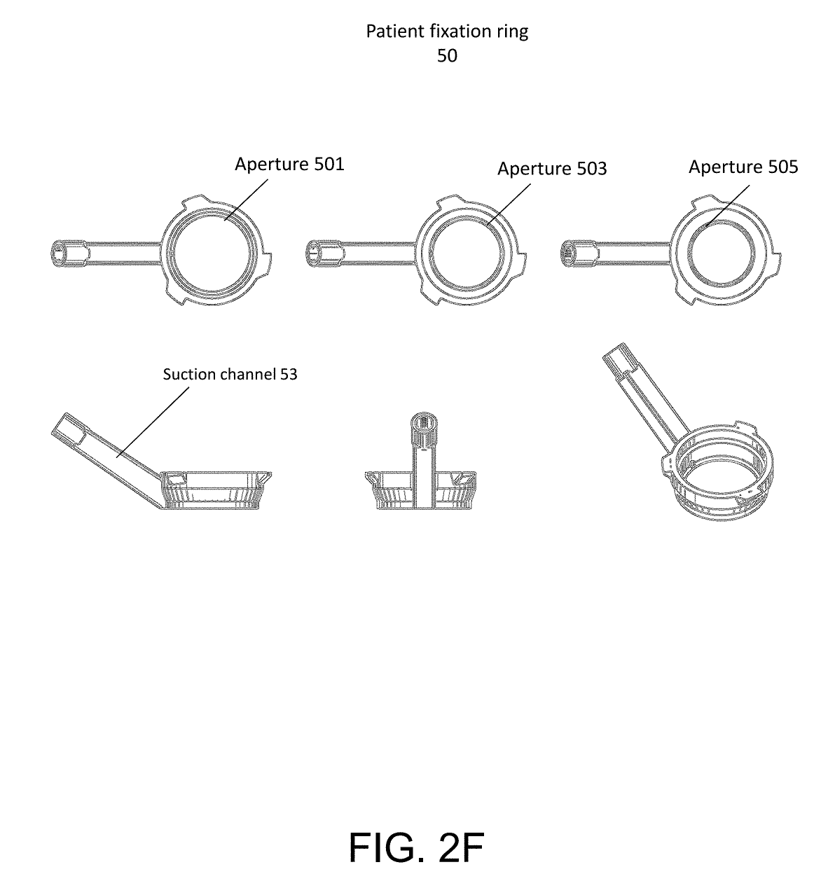

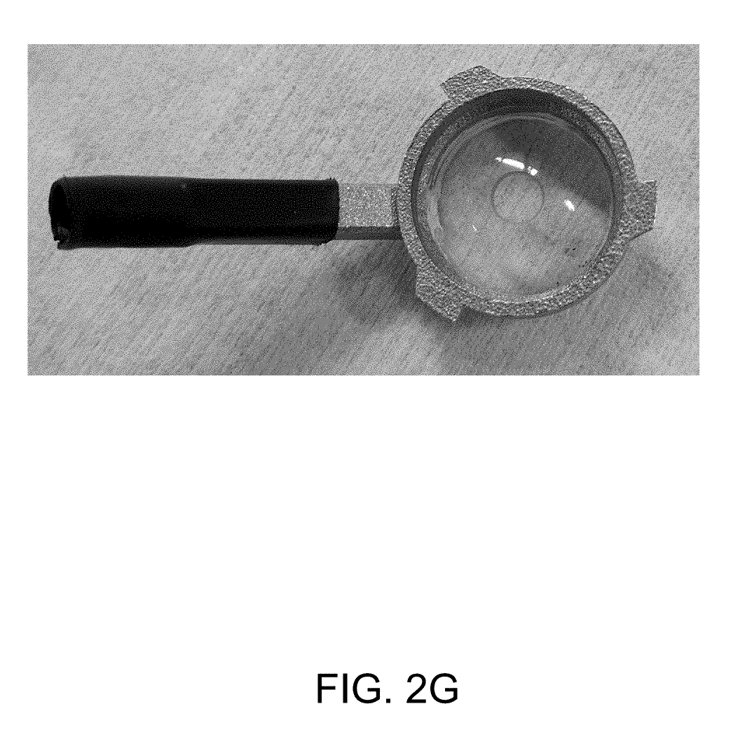

The contact lens device is well suited from combination with prior patient interface devices that are used to hold the eye steady during surgery. FIG. 2A is originally from U.S. Pat. No. 6,899,707, entitled “APPLANATION LENS AND METHOD FOR OPHTHALMIC SURGICAL APPLICATIONS,” the entire contents of which are incorporated herein by reference. Such patient interfaces may be well suited for use with scleral translocation elasto-modulation methods and apparatus, and can be combined with the patient interface incorporated by reference above and the contact lens device as described herein.

FIG. 2A shows an exploded view of an exemplary ocular fixation system 10. The ocular fixation system 10 may be an apparatus that attaches to an eye or near an eye and may hold (fix) the eye in all three axes (x, y and z) from translational and rotational movement with respect to a beam of a laser surgical device. In addition, the ocular fixation system 10 allows for the eye to be treated by a lens 18 (such as those described in FIG. 1A-1G), laser optic (not illustrated), or laser (not illustrated), or any combination thereof, for ophthalmic surgery. The ocular fixation system 10 may grip, hold or affix the eye to the lens 18 or laser optic, during a laser surgical procedure, so as to minimize or preclude differential motion of the human eye with respect to the laser optical path during the laser procedure.

The ocular fixation system 10 may comprise an ocular attachment ring 12 that may couple the ocular fixation system 10 to the eye, a gripper fixture 14 to aid a user in placement or positioning, a lens cone fixture 16, and a lens 18 of any type described herein.

The component parts of the ocular fixation system 10 are illustrated in an exploded view, and are intended to be collapsed vertically, such that each of the individual portions of the device are in mechanical engagement with appropriate other portions, such that the completed device may be provided in a generally unitary structure. However, the device's component parts need not be permanently affixed to one another; the component parts may be separable and interchangeable. In some embodiments, the ocular fixation system 10 may form a single composite interface structure between a human eye and a surgical laser once the component parts have been aligned with a patient's eye and with respect to the laser delivery system.

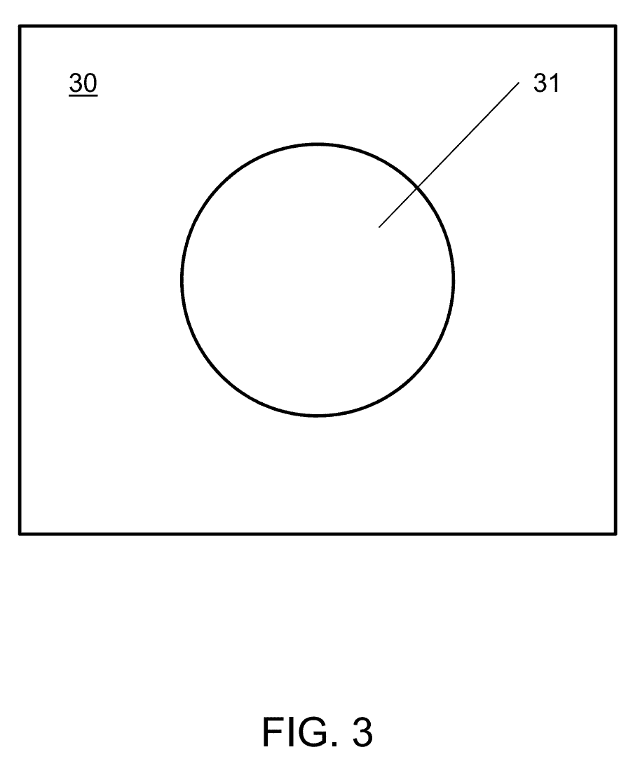

The system 10 may be compatible with various sterilization procedures such that sterilization does not significantly alter the optical transmissive, heat conductive, or electrical conductive properties. For instance, the lens 18 may be subjected to a gamma raditation sterilization process (e.g., 25 kGy-40 kGy). The system 10 may be sterilized separately from the lens 18. The lens may come sterilized in a kit (as shown in FIG. 3).

FIGS. 2B-2G show examples of an ocular fixation system 20 for coupling an energy source to a surface of an eye. FIG. 2B shows a side view of a structure for coupling an energy source to a surface of an eye. FIG. 2C shows a side view of a structure for coupling an energy source to a surface of an eye and a top view of a structure for coupling an energy source to a surface of an eye. FIG. 2D shows an exemplary cone structure 40 and a patient fixation ring structure 50. FIG. 2E shows an exemplary cone structure 40 and with exemplary laser support structure 42. FIG. 2F shows exemplary patient fixation ring structures with different aperture dimensions. FIG. 2G shows an example of a clear contact lens assembled to a patient fixation ring structure.

The ocular fixation system 20 may comprise a cone structure 40. The cone 40 may be configured to remove heat from a surface of an eye. The cone may be composed of a material having a high thermal conductivity. For example, the cone may comprise graphene to improve thermal conductivity. The cone may comprise graphene in a range of about 5% to 80%. The cone structure may be constructed of a substantially rigid material such as a rigid, extruded plastic, aluminum, or the like. In some cases, the cone may be a metal cone.

The cone 40 may be coupled to a laser support structure 42 and an optics tray 43. The laser support structure may support one or more laser sources, as described herein. FIG. 2E shows an example of a laser support structure 42. In the example, the laser support structure may comprise one or more channels accommodating one or more optical fibers or light source cables. The optics tray 43 may support one or more optical components that direct one or more lasers to a surface of an eye, as described herein. In some examples, one or more optical components such as optic fibers may be configured to illuminate iris and corneo-scleral limbus. One or more optical components may be embedded in the wall of the cone structure or located in various other locations in the cone structure to provide illumination to the eye. The one or more optical components may provide illumination such as white light or near-infrared light.

The cone 40 may be coupled to a patient fixation ring 50. The cone 40 may comprise a laser support structure 42 as described above. The patient fixation ring 50 may be configured to form an air-tight seal with a surface of the cone. The patient fixation ring may be coupled to a surface of the cone using a compression fitting 58. In some embodiments, the patient fixation ring may comprise a snap-fit feature to be coupled to a complementary feature of the cone. The snap-fit feature allows the patient fixation ring to be releasably connected to the cone in an easy and convenient manner. As shown in FIG. 2D, the cone 40 may comprise a snap-fit feature 57 matched to a snap-fit feature 59 of the patient fixation ring 50 to form a compression fitting. In some embodiments, a bottom portion 41 of the cone 40 may be in contact with and press against the contact lens when the cone 40 is assembled to the patient fixation ring. The bottom portion 41may have an aperture that may be smaller than the bottom aperture of the patient fixation ring. The bottom portion may be partially or totally received by the patient fixation ring when in an assembled configuration. The patient fixation ring may be configured to provide suction. For instance, the patient fixation ring may be coupled to suction tubing 52. The patient fixation ring may comprise a suction channel 53 in fluidic communication with the suction tubing 52. The suction may be provided by connecting a syringe to the suction tubing and withdrawing the syringe. The suction tubing 52 may comprise a vacuum pressure sensor. Additionally or alternatively, the vacuum pressure sensor may be provided at the suction channel 53. The pressure sensor may be used to determine that the coupling structure is properly connected to an eye.

The patient fixation ring 50 forms the mechanical interface between the anterior surface of a human cornea and the cone. The patient fixation ring 50 may be constructed of a flexible, hypoallergenic material such as rubber, hypoallergenic plastic, silicone, or the like. The patient fixation ring 50 may be constructed of a rigid material such as polymer.

The patient fixation ring may be coupled to a heat sink contact lens 54. The heat sink contact lens 54 may be seated within the patient fixation ring 50. The hear sink contact lens may be the same as the lens as described in any of FIGS. 1A-1G. The heat sink contact lens may be composed of a material having a high thermal conductivity. The heat sink contact lens may be composed of sapphire. The heat sink contact lens may be composed of diamond or a diamond-like material. The heat sink contact lens may have an outer diameter of about 15 mm, 16 mm, 17 mm, 18 mm, 19 mm, or 20 mm. The heat sink contact lens may have an outer diameter of less than 15 mm. The heat sink contact lens may have an outer diameter of greater than 20 mm. The heat sink contact lens may have a thickness of about 0.5 mm, 1 mm, or 1.5 mm. The heat sink contact lens may have a thickness of less than 0.5 mm. The heat sink contact lens may have a thickness of greater than 1.5 mm.

The heat sink contact lens may comprise a hole 56 located at approximately the center of the heat sink contact lens. The hole 56 may allow for the flow of fluids (such as air) away from the eye.

The cone 40 may be positioned on a counter-weighted moveable arm such that no weight rests on the eye when the cone is docked to the patient fixation ring. The cone 40 may have a fixed working distance such that the distance between the surface of the eye and the energy source may be constant between patients. The cone may be thermally controlled, for example with a fluid-based (such as water-based) heat exchanger or Peltier cooler, in order to help maintain the desired temperature of the patient interface and/or contact lens. Controlling the temperature of the cone may allow the preservation of tissues within the eye during an interaction with an energy source. For instance, cooling the cone may allow for the preservation of the epithelium during heating with a laser source.

In some embodiments, the cone 40 may be thermally controlled using a fluid-based heat exchanger. The cone may comprise one or more fluid channels. The fluid channels may be fluidically coupled to a chiller 46 through one or more couplings and one or more tubes or hoses. The couplings 48 may be threaded couplings. The couplings 48 may be compression couplings. The chiller 46 may circulate a cooling fluid (such as water, ethylene glycol, or another liquid coolant) through the fluid channels in order to cool the cone. The chiller 46 may cool the cone to a temperature less than 37° C., less than 30° C., less than 25° C., less than 20° C., less than 15° C., less than 10° C., less than 5° C., or less than 0° C. The chiller may comprise a water exchanger. The water exchanger may have a lateral footprint of approximately 8 inches×8 inches. The water exchanger may draw an electric power of approximately 160 W.

In some embodiments, the cone 40 may be thermally controlled using a thermoelectric cooler. The thermoelectric cooler may comprise a Peltier cooler. The Peltier cooler may be placed in thermal connection with the heat sink lens. The Peltier cooler may be located on the counter-weighted moveable arms. The Peltier cooler may cool the cone to a temperature less than 37° C., less than 30° C., less than 25° C., less than 20° C., less than 15° C., less than 10° C., less than 5° C., or less than 0° C.

The system may be operated by applying vacuum to the eye, aiming an illumination beam at the eye, and obtaining an OCT image of the eye. The OCT image may provide a baseline image of the eye prior to treatment. A treatment may be started once the heat sink contact lens has been secured in place by the vacuum. An OCT image may be obtained following treatment. The OCT image may be compared with the baseline image to obtain a precise measurement of changes induced by the treatment.

With regard to the laser delivery system, it will be understood that the lens cone fixture 40 may be adapted to be affixed to the distal end of a laser optical delivery system, such that the delivery system need only be concerned with focusing an incident laser beam at a particular point in space. The laser delivery system may be disposed at a specific distance from the interface between the base ring and the laser delivery system, such that the anterior surface of the eye, or at least that portion in contact with the lens, is at a known specific distance from the laser delivery tip. In some cases, the laser delivery system may have deliver energy (e.g., light or electromagnetic energy) with a wavelength from about 800 micrometers to about 1.5 millimeters. In some cases, the laser delivery system may have deliver energy (e.g., light or electromagnetic energy) with a wavelength from about 1.5 millimeters to about 2.4 millimeters. In some cases, the laser delivery system may have deliver energy (e.g., light or electromagnetic energy) from about 1.8 millimeters to about 2.2 millimeters. In some cases, the laser delivery system may have deliver energy (e.g., light or electromagnetic energy) with a wavelength from about 4 millimeters to about 7 millimeters. In some cases, the laser delivery system may have deliver energy (e.g., light or electromagnetic energy) from about 5 millimeters to about 7 millimeters. In some cases, the laser delivery system may have deliver energy (e.g., light or electromagnetic energy) with a wavelength of about 6 millimeters. In some instances, the laser delivery system may have deliver energy (e.g., light or electromagnetic energy) having a wavelength equal to about 0.4 um, 0.6 um, 0.8 um, 1 um, 1.2 um, 1.4 um, 1.6 um, 1.8 um, 2.0 um, 2.2 um, 2.4 um, 2.6 um, or any value therebetween. The laser may of any type known in the art, such as a CO₂-based laser. The laser may comprise one or more of many lasers emitting one or more of many wavelengths, such as infrared lasers. In many embodiments, the laser may comprise a quantum cascade laser configured to emit light having a wavelength within a range from about 5.8 to about 6.6 um, for example from about 6 to about 6.25 um. In many embodiments, the laser may comprise a quantum cascade laser or continuous wave laser configured to emit light having a wavelength within a range from about 1 to about 6 um, such as from about 1 to 3 um. In many embodiments the laser is configured to emit light having a wavelength within a range from about 1.4 to about 2 um, for example about 1.46 um or 2.01 um, and other wavelength ranges as described herein. Such lasers are commercially available, and can be configured by a person of ordinary skill in the art for treatment of the eye as described herein.

FIG. 2F shows exemplary patient fixation rings 50 with different sizes of bottom apertures 501, 503, 505 to accommodate different sizes of contact lenses. The bottom aperture of the fixation ring structure may have various different diameters to accommodate various different diameters of contact lenses. For example, the bottom aperture may have an inner diameter of about 15 mm, 16 mm, 17 mm, 18 mm, 19 mm, or 20 mm. The bottom aperture may have an inner diameter of less than 15 mm. The bottom aperture may have an inner diameter of greater than 20 mm. In the illustrated example, three different diameters 15 mm, 17 mm, and 19 mm are shown to fit the dimensions the contact lenses with outer diameters 15 mm, 17 mm, and 19 mm. Patient fixation rings with different bottom apertures may or may not have the same top apertures to accept the cone structure. The patient fixation ring may comprise one or more suction channels 53 to provide suction. For instance, the suction channel of the patient fixation ring may be coupled to a suction tubing 52. The suction may be provided by connecting a syringe to the suction tubing and withdrawing the syringe. The suction tubing 52 may comprise a vacuum pressure sensor. Additionally or alternatively, the vacuum pressure sensor may be provided at the suction channel 53. The pressure sensor may be used to determine that the coupling structure is properly connected to an eye.

FIG. 2G shows an example of a transparent contact lens assembled to a patient fixation ring structure. In some embodiments, the contact lens may be optically transmissive or visually translucent, whereby at least a portion of light is permitted to pass through the contact lens to allow at least partial visualization through the contact lens. For example, a transparent contact lens or a translucent contact lens may allow most of the light in the visible spectrum to pass through and allow at least partial visualization through the contact lens. In some embodiments, the contact lens may be semi-transparent or semi-translucent that may allow only a portion of the visible light or certain wavelengths of light to pass through, thereby resulting in visibility being reduced to some extent. The contact lens may be at least partially transparent to the visible light spectrum, such that a doctor or physician can see through the contact lens to view the underlying patient eye.

In some cases, the provided contact lens or system may be used in a surgical procedure using an energy source comprising a high intensity focused ultrasound (HIFU). In some cases, the energy source may comprise an amount of energy per unit time (power) to the eye within a range from about 50 mW to about 900 mW. In some cases, the energy source may comprise an amount of energy per unit time (power) to the eye within a range from about 100 to about 700 mW. In some cases, the energy source may comprise an amount of energy per unit time (power) to the eye within a range from about 200 to 400 mW. The energy source may comprise one or more of a pulsed laser, a continuous laser, a pulsed ultrasound transducer, a HIFU array, or a phased HIFU array.

FIG. 3 shows a kit 30 comprising a lens with layers as described herein contained within a sterile packaging. The sterile packaging may ensure that the lens is sterile prior to use. Sterilization of the lens 31 or the kit 30, or both, may be done with any method known in the art. In some embodiments, the lens may be single use lens. Alternatively, the lens may be configured to be used repeatedly. For example, the lens may be configured to be used one, two, three, four, five, six, seven, eight, nine, ten, or more times. Optionally, the lens may be configured to be used by a single individual over periods of time.

In some cases, the lens may comprise information to be read by a device to automatically set up a laser surgery parameters to be compatible with the lens. For instance, the lens or the lens kit may comprise a barcode configured to be read by a reader. The barcode may encode information such as an identifier of the contact lens, specification of the contact lens, suggested surgery parameters and/or various other information related to the lens or the surgery using the lens.

The barcode may define elements such as the version, format, position, alignment, and timing of the barcode to enable reading and decoding of the barcode. The remainder of the barcode can encode various types of information in any type of suitable format, such as binary or alphanumeric information. The barcode may be two dimensional such as PDF417, Aztec, MaxiCode, and QR code, etc. The barcode may be one-dimensional barcode such as Interleaved 2/5, Industrial 2/5, Code 39, Code 39 Extended, Codabar, Code 11, Code 128, Code 128 Extended, EAN/UCC 128, UPC-E, UPC-A, EAN-8, EAN-13, Code 93, Code 93 Extended, DataBar Omnidirectional (RSS-14), DataBar Truncated (RSS-14 Truncated), DataBar Limited (RSS Limited), DataBar Stacked, DataBar Expanded, DataBar Expanded Stacked, etc. The barcode can encode various types of information in any type of suitable format, such as binary, alphanumeric, ASCII, etc., and the code can be based on any standards. The barcode may be read by an optical reader, laser scanner or other imaging device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A lens for conducting heat away from an eye during laser eye surgery, said lens comprising:
a first surface for contacting the eye;
a second surface for receiving light energy, wherein the lens comprises graphene between the first surface and the second surface and wherein the lens is configured to transmit light with a transmittance within a range from about 50% to 99%.

2. The lens of claim 1, wherein the first surface comprises an external surface of a first layer of graphene or graphene like material and the second surface comprises an external surface of a second layer of graphene or graphene like material and wherein a polymeric substrate layer is located between the first layer and the second layer and optionally wherein the light transmittance is within the range from about 400 nanometers (nm) to about 2200 nm and optionally wherein the polymeric substrate layer comprises acrylate.

3. The lens of claim 1, wherein the lens comprises a layer comprising graphene between the first surface and the second surface.

4. The lens of claim 3, wherein the layer comprising graphene comprises the first surface and the second surface and optionally wherein the first surface and the second surface comprise outer surfaces of the lens, the first surface comprising a posterior surface and the second surface comprising an anterior surface.

5. The lens of claim 3, wherein the layer comprises a cured mixture of graphene and an optically transmissive material located between the first surface and the second surface.

6. The lens of claim 5, wherein second surface comprises a surface of the cured mixture.

7. The lens of claim 5, wherein the cured mixture comprises a percentage of graphene within a range from about 1% to about 50% by weight and a percentage of optically transmissive material within a range from about 99% to about 50%.

8. The lens of claim 5, wherein the cured mixture of graphene comprises a matrix comprising graphene particles dispersed in the optically transmissive material and optionally wherein the graphene particles are dispersed in the optically transmissive material with a uniformity of +/−5%.

9. The lens of claim 5, wherein the cured mixture of graphene comprises a matrix comprising graphene particles dispersed in the optically transmissive material and wherein an absorbance of the matrix varies by no more than +/−5% for a light beam transmitted through the matrix.

10. A method of conducting heat away from an eye in laser eye surgery, said method comprising:
coupling a lens to an eye, said lens comprising:
a first surface for contacting the eye;
a second surface for receiving light energy, wherein the lens comprises graphene between the first surface and the second surface and wherein the lens is configured to transmit light with a transmittance within a range from about 50% to 99%;
directing energy to the eye using a laser; and
conducting heat away from the eye via the graphene between the first surface and the second surface.

11. The method of claim 10, wherein the lens comprises a layer comprising graphene between the first surface and the second surface.

12. The method of claim 11, wherein the layer comprising graphene comprises the first surface and the second surface and optionally wherein the first surface and the second surface comprise outer surfaces of the lens, the first surface comprising a posterior surface and the second surface comprising an anterior surface.

13. The method of claim 11, wherein the layer comprises a cured mixture of graphene and an optically transmissive material located between the first surface and the second surface.

14. The method of claim 13, wherein second surface comprises a surface of the cured mixture.

15. The method of claim 13, wherein the cured mixture comprises a percentage of graphene within a range from about 1% to about 50% by weight and a percentage of optically transmissive material within a range from about 50% to about 99%.

16. The method of claim 13, wherein the cured mixture of graphene comprises a matrix comprising graphene particles dispersed in the optically transmissive material and optionally wherein the graphene particles are dispersed in the optically transmissive material with a uniformity of +/−5%.

17. The method of claim 13, wherein the cured mixture of graphene comprises a matrix comprising graphene particles dispersed in the optically transmissive material and wherein an absorbance of the matrix varies by no more than +/−5% for a light beam transmitted through the matrix.
